(19) United States
(12) Patent Application Publication
(10) Pub. No.: US 2020/0108241 A1
(43) Pub. Date: Apr. 9, 2020

(54) Systems and Methods for Delivering Exosomes Through the Blood-Brain Barrier

(71) Applicant: Synaptec Network, Inc., Santa Monica, CA (US)

(72) Inventor: Sheldon Jordan, Pacific Palisades, CA (US)

(21) Appl. No.: 16/592,211

(22) Filed: Oct. 3, 2019

Related U.S. Application Data
(60) Provisional application No. 62/741,365, filed on Oct. 4, 2018.
(60) Provisional application No. 62/829,862, filed on Apr. 5, 2019.

(51) Int. Cl. A61M 37/00; A61N 5/06; A61K 35/51

(52) U.S. Cl. CPC A61M 37/0092; A61N 5/062; A61K 35/51; A61N 2007/0039; A61M 2210/0693; A61N 2005/0659; A61M 2205/052

(57) ABSTRACT

Methods, systems, and devices are disclosed for therapeutic stimulation of targeted regions of the brain, improved delivery of exosomes across the blood brain barrier, or combinations thereof. An ultrasound transducer is used to target a region of therapeutic interest in a patient's brain. The transducer sonicates the targeted region, forming openings in the blood brain barrier or increasing local perfusion at the targeted region. Near-infrared light sources can further be applied to the targeted region to increase delivery of exosomes, either in combination with sonic transducers. In some embodiments, near-infrared light sources are applied to the targeted region to regulate neurobiological function or encourage neurotherapeutic effects, either alone or in combination with the administration of exosomes or therapeutic agents to the patient.

This application claims priority to U.S. provisional application 62/829,862, filed Apr. 5, 2019, and U.S. provisional application 62/741,365, filed Oct. 4, 2018, the disclosures of which is incorporated herein by reference in the entirety.

### FIELD OF THE INVENTION

The field of the invention is methods, systems, kits, and devices related to delivering exosomes to the brain.

### BACKGROUND

The background description includes information that may be useful in understanding the present invention. It is not an admission that any of the information provided herein is prior art or relevant to the presently claimed invention, or that any publication specifically or implicitly referenced is prior art.

In some cases, the most desirable way to treat an ailment is to treat the source. However, for treatment of ailments in or relating to regions of the brain, the blood-brain barrier (BBB) often hinders treatment by preventing diffusion of therapeutic agents into the brain. While it is known to mechanically bypass the BBB, for example through use of a needle to inject therapeutic agents directly into the brain, such methods are undesirable due to damage caused by such invasive methods.

More sophisticated methods of delivering therapeutic agents to regions of the brain are known. For example, “Targeted Delivery of Neural Stem Cells to the Brain Using MRI-Guided Focused Ultrasound to Disrupt the Blood-Brain Barrier,” PLoS ONE 6(11): e27877. doi:10.1371, by Burgess, et al. reports using MM guided focused ultrasound with microbubbles to temporarily open targeted regions of the BBB to allow entry of neural stem cells in animal models. Similarly, “Cellular Mechanisms Of The Blood-Brain Barrier Opening Induced By Ultrasound In Presence Of Microbubbles,” Ultrasound in Med. & Biol., Vol. 30, No. 7, pp. 979-989, 2004 by Sheikov, et al reports using ultrasound and microbubbles in animal models to open the BBB, but cautions tissue in the BBB can be damaged at 3 W sonications. Likewise, “Noninvasive Localized Delivery Of Herceptin To The Mouse Brain By Mri-Guided Focused Ultrasound-Induced Blood-Brain Barrier Disruption,” PNAS, Vol. 103, No. 31, 11719-23 by Kinoshita, et al reports using ultrasound with microbubbles in animal models to disrupt BBB and deliver Herceptin (150 kDa) across the BBB, and notes the presence of microbubbles is required for consistent BBB opening. But it does not appear known to deliver exosomes across the BBB, to use ultrasound to open the BBB in humans, or to use ultrasound to safely and temporarily open the BBB in the absence of microbubbles.

Similarly, recent studies have emerged with a focus on transcranial infrared laser stimulation in humans and its effects on emotional and cognitive functioning. Transcranial brain stimulation by low-level light/laser therapy uses directional light from lasers or LEDs in the red-to-near-infrared wavelengths to promote a variety of biological effects, including the enhancement of energy production, gene expression, and the prevention of cell death (Rojas, J. C. & Gonzalez-Lima, F., Neurological and psychological applications of transcranial lasers and LEDs. Biochemical Pharmacology. 86, 447-457 (2013); see also Hamblin, M. R., Shining light on the head: Photobiomodulation for brain disorders. BBA Clinical, 6, 113-124 (2016)). However, there appears to be a lack of appreciation for therapeutic methods either combining transcranial infrared stimulation with exosome delivery across the BBB, or improved standalone therapeutic applications of near-infrared transcranial stimulation.

All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Thus, there remains a need for systems and methods for delivering exosomes to the brain in humans or to otherwise to regulate neurobiological function or encourage neurotherapeutic effects in a patient in a non-thermal, nondestructive manner.

### SUMMARY OF THE INVENTION

The inventive subject matter provides apparatus, systems, and methods for delivering an exosome across the blood-brain barrier (BBB) of a (preferably human) patient. A region of the patient's brain, preferably a region of potential therapeutic interest, is targeted with a sonic transducer. The sonic transducer is used to sonicate the targeted region of the brain, which improves the targeted region by facilitating selective delivery of the exosome to the targeted region, for example by opening the BBB, increasing local perfusion in the targeted region, etc. While opening (e.g., temporarily, reversibly, both) the BBB or increasing local perfusion in the targeted region (or both) are preferred means of facilitating delivery of exosomes or therapeutics to the brain, it should be appreciated that other means are contemplated that mitigate damage to the BBB or surrounding brain tissue while promoting delivery of therapeutics or exosomes. The exosome is administered to the patient's blood stream, preferably after ultrasound treatment, and the exosomes are preferentially delivered to the target in the brain.

The inventive subject matter further provides apparatus, systems, and methods for transcranial brain stimulation using light therapy to regulate neurobiological function or encourage neurotherapeutic effects in a patient in a non-thermal, nondestructive manner. One or more directional, low-power, and high-fluency monochromatic laser or LED light sources (in some embodiments both) are used to direct light with red-to-near-infrared wavelengths at a target region (or regions) of the patient's brain. The red-to-near-infrared light can be continuous, pulsing, or alternating, and is used to regulate neurobiological function in the targeted region(s) or encourage neurotherapeutic effects in a non-thermal, nondestructive manner. It is contemplated such treatment can promote a variety of biological effects, including the enhancement of energy production, gene expression, and the prevention of cell death. In some embodiments, exosomes or other therapeutic agents are delivered in conjunction (e.g., before, after, during, alternating, overlapping, etc.) with light treatment.

### BRIEF DESCRIPTION OF THE DRAWINGS

FIG. 1 shows a flow chart of a method of the inventive subject matter.

FIG. 2 shows a flow chart of another method of the inventive subject matter.

### DETAILED DESCRIPTION

The inventive subject matter provides apparatus, systems, and methods for delivering an exosome across the blood-brain barrier (BBB) of a (preferably human) patient. A region of the patient's brain, preferably a region of potential therapeutic interest, is targeted with a sonic transducer. The sonic transducer is used to sonicate the targeted region of the brain, which improves the targeted region by facilitating selective delivery of the exosome to the targeted region, for example by opening the BBB, increasing local perfusion in the targeted region, etc. While opening (e.g., temporarily, reversibly, both) the BBB or increasing local perfusion in the targeted region (or both) are preferred means of facilitating delivery of exosomes or therapeutics to the brain, it should be appreciated that other means are contemplated that mitigate damage to the BBB or surrounding brain tissue while promoting delivery of therapeutics or exosomes. The exosome is administered to the patient's blood stream, preferably after ultrasound treatment, and the exosomes are preferentially delivered to the target in the brain.

The region of the patient's brain to be targeted is preferably associated with a disease condition. In some embodiments, the disease condition is associated with at least one of dementia, a learning disorder, an anxiety disorder, a motor disorder, a consciousness disorder, a movement disorder, an attention disorder, a stroke, a vascular disease, Alzheimer's disease, Parkinson's disease, multiple sclerosis, cancer, schizophrenia, depression, substance abuse, and traumatic brain injury. However, any disease or disease condition that is pathologically associated with a region of the brain is appropriate for the contemplated methods. For example, the targeted region of the patient's brain can be the frontal lobe, parietal lobe, occipital lobe, temporal lobe, hippocampus, hypothalamus, brain stem, cerebellum amygdala, corticospinal tract, thalamus, substantia nigra, basal ganglia, a tumor, a lesion, necrotic tissue, Heschl's gyms, Brodmann area 25, a point of injury, or any other region of interest. In some embodiments more than one region of the brain is targeted, for example to treat more than one disease or to combat a disease associated with more than one region of the brain.

In some embodiments, the exosome is derived from full (or partial) term placental tissue, umbilical tissue, mesenchymal stem cell, is derived synthetically, or some combination thereof. For example, exosomes could be cultured in a supernatant solution, with the solution containing the exosomes administered to the patient. Viewed from another perspective, the administration of exosomes to the patient includes the administration of exosomes or media (e.g., fluids, solutions, plasmas, infusions, powders, inhalants, dehydrates, etc.) comprising, containing, or conveying the exosomes, or combinations thereof. In some embodiments it is favorable to deliver more than one exosome across the BBB of the patient, in some cases delivering multiple exosomes of multiple types or derivations. The exosomes can also contain therapeutic agents (e.g., medication, protein, antibody, etc), whether native, engineered, or synthetic.

The sonic transducer is preferably an ultrasonic transducer, though infrasonic and audible transducers are also contemplated. The ultrasonic transducer preferably has a working frequency of at least 20 Hz, more preferably at least 200 Hz, at least 1 MHz, or at least 5 MHz. In some embodiments, working frequencies of more than 1 GHz are used. Likewise, the sonic transducer is operated at a power of at least 100 mW/cm², though powers of more than 200 mW/cm², more than 400 mW/cm², and more than 800 mW/cm² are also contemplated. Typically, the region of the patient's brain is sonicated for at least 1 minute, sonication can last 10 minutes, 20 minutes, 30 minutes, or more than 45 minutes, and optionally include pulses of 1 second, 5 seconds, 10 seconds, or more than 30 seconds. In some embodiments, therapeutic agents are also administered to the patient's blood stream, whether before, after, or during sonication of the targeted region of the brain.

In some embodiments, a microbubble, or plurality thereof, is also administered to the patient's blood stream. In such embodiments, the sonic transducer is optionally used to sonicate the microbubble, preferably when the microbubble is proximal to the region of the patient's brain. In some embodiments, the microbubbles contain a therapeutic agent.

The inventive subject matter further provides apparatus, systems, and methods for transcranial brain stimulation using light therapy to regulate neurobiological function or encourage neurotherapeutic effects in a patient in a non-thermal, nondestructive manner. Laser or LED light sources (in some embodiments both) are used to direct light with red-to-near-infrared wavelengths at a target region of the patient's brain. The red-to-near-infrared light can be continuous, pulsing, or alternating, and is used to regulate neurobiological function in the targeted region(s) or encourage neurotherapeutic effects in a non-thermal, nondestructive manner.

In some embodiments, light treatment is also applied to the targeted region of the BBB to further improve delivery of therapeutics or exosomes across the BBB, either as an alternative to acoustic waves (e.g., ultrasound) or in combination with acoustic treatment (e.g., sequentially, simultaneously, in a pattern, etc). Viewed from another perspective, light (e.g., indirect light, direct light) can further be applied to a targeted region of the BBB to open the BBB (preferably temporary, reversibly, or both) or to increase local perfusion at the targeted region of the BBB or at a different desired point of perfusion, in addition to or as an alternative to transcranial brain stimulation by light therapy.

Preferred light treatments include application of infrared or near infrared light, for example lasers in either spectrum of no more than 0.5 J/cm², 1 J/cm², 5 J/cm², 10 J/cm², 15 J/cm², 20 J/cm², 25 J/cm², 30 J/cm², 35 J/cm², 40 J/cm², 50 J/cm², or 75 J/cm². Similarly, lasers with overall power less than 10 W, 20 W, 30 W, 40 W, 50 W, 60 W, 70 W, or less than 100 W are contemplated, preferably selected such that less than 25%, 20%, 15%, 10%, 5%, or less than 3%-2% of the laser power or intensity reaches the brain (e.g., after passing through skin, skull, tissue, etc). Likewise, the light (e.g., laser) can be continuous or pulsed, for example pulsed at a frequency of 1 mHz, 10 mHz, 100 mHz, 1 Hz, 10 Hz, 100 Hz, 1 kHz, 10 kHz, 100 kHz, 1 MHz, 10 MHz, or 100 MHz, or a range therein. The laser (or plurality of lasers) is applied for a duration of 1 ms, 10 ms, 100 ms, 1 s, 10 s, 30 s, or 60 s, either continuously or pulsed. While infrared or near infrared spectra are preferred, it is contemplated that UV, x-ray, gamma ray, radar, or radio wave spectra can also be directed at the BBB to improve delivery of therapeutics or exosomes, either separately or in combination (e.g., sequentially, simultaneously, etc) with other spectra or acoustic waves (e.g., ultrasonic).

Further, while focused light (e.g., lasers) is preferred, diffused light can favorably be applied to aid in delivery of exosomes through the BBB. For example, low energy, diffuse light (of one or more spectra) can be applied to a targeted region of the BBB in combination with high intensity focused ultrasound in order to temporarily or reversibly, or both, open the BBB at the targeted region or increase local perfusion. Similarly, high energy and high focused light (e.g., laser of one or more spectra) can be applied to a targeted region of BBB in conjunction with low intensity, diffuse sonic treatment (e.g., ultrasound), to reversibly or temporarily (or both) open the BBB or increase local perfusion at the target.

More than one light source can be applied to the targeted region to improve therapeutic stimulation (e.g., improve regulation of neurobiological function, improve neurotherapeutic effects, etc.), to improve delivery of a therapeutic agent or exosome across the BBB, or some combination thereof. For example, lights of different wavelengths, or pulsed with the same or different frequencies, can be directed at a targeted area of the brain or the BBB. It is contemplated that the wavelength or amplitude of more than one light source can be selected to constructively interfere at the targeted region of the brain or BBB, to deconstructively interfere en route to the targeted region of the BBB (e.g., through flesh, skull, non-target brain matter, etc), or some combination thereof.

Therapeutic methods and methods of treating a patient by delivering an exosome to a region of the patient's brain via (i) a temporary opening in the patient's BBB or (ii) increasing perfusion local to the region are also contemplated. A sonic transducer is preferably used to form the temporary opening or increase local perfusion.

While it is contemplated the inventive subject matter is applicable to any condition (e.g., disease, disorder, characteristic, etc) and region of the brain, preferred conditions for treatment, and regions of the brain for targeting, include those listed in Table 1.

TABLE 1

| Condition | Region of the Brain |
|---|---|
| Alzheimer's disease: | Hippocampus and surrounding cortex |
| Parkinson's disease: | Substantia nigra and basal ganglia |
| Vascular dementia: | Diffusely throughout the brain |
| MS: | Proximal to MS lesions |
| Cancer: | Proximal to tumor and necrotic tissue |
| Schizophrenia: | Frontal lobe and Heschl's gyrus |
| Depression: | Frontal lobe and Brodmann area 25 |
| Substance abuse: | Diffusely throughout the cortex but likely not in |
| | subcortical structures |
| Traumatic Brain | Proximal to area of injury |
| Injury: | |

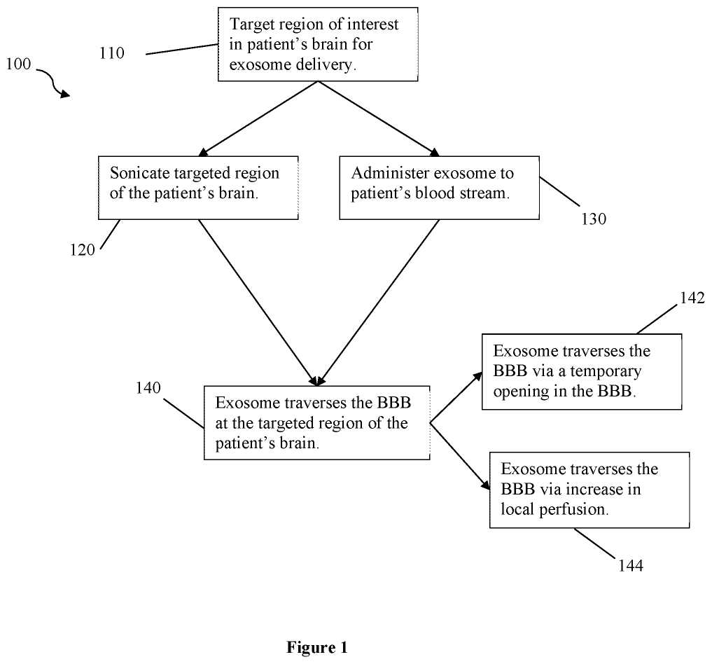

FIG. 1 depicts flow chart 100 of a therapeutic method of the inventive subject matter. In step 110, a region of the patient's brain is targeted with a sonic transducer for exosome delivery, preferably an ultrasonic transducer. In some cases more than one sonic transducer is targeted at a single region of the patient's brain, but it is also contemplated that multiple sonic transducers are directed toward more than one targeted region of the patient's brain. Preferably, the region is targeted using neuronavigation, for example based on Mill data specific to the patient. In step 120, the sonic transducer is used to sonicate the targeted region of the patient's brain. Step 130 administers an exosome (typically plurality of same type of exosome) to the patient's blood stream.

Step 140 occurs after step 130 has administered the exosome(s) to the patient. In step 140, the exosome(s) traverses the BBB at the targeted region of the patient's brain. It is contemplated that the exosome(s) traverses the BBB either by step 142, the formation of holes in the BBB at the targeted region, or by step 144, the increase of local perfusion at the targeted region, or both. For example, the performance of step 120 can be tuned to both form holes in the BBB and increase local perfusion without damaging the BBB, such as by use of multiple transducers with varying frequencies, periodicity, and intensity directed to one or more targeted regions of the brain. For example, step 120 can be performed to form holes in the BBB in one targeted region, while increasing local perfusion at another targeted region.

Steps 120 and 130 can also be performed substantially simultaneously, separately (e.g., step 120 first, step 130 following, vice versa, etc.), or in an alternating pattern (e.g., step 120 followed by step 130, followed by periodic on/off repeat of step 120). In preferred embodiments, step 120 is repeated periodically at low intensity to prevent damage to the BBB or undesired increase in local perfusion in the target region. Viewed from another perspective, step 120 is performed to form holes in the BBB large enough for the exosome to traverse the BBB while avoiding damage to the BBB, for example preserving the ability of the BBB to close after sonicating is ceased.

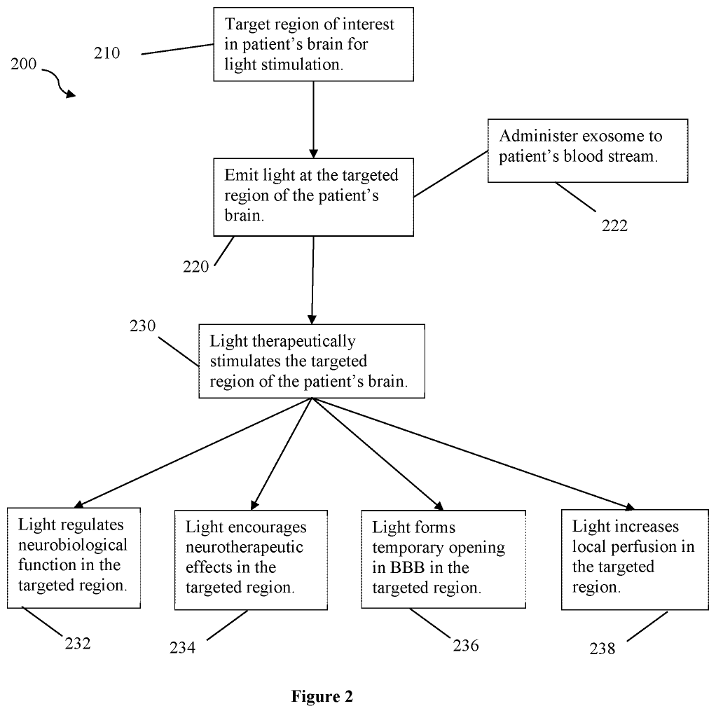

FIG. 2 depicts flow chart 200 for another method of the inventive subject matter. In step 210, a region of the patient's brain is targeted for light stimulation, for example targeting a laser or LED with red or near-infrared light by neuronavigation based on Mill data specific to the patient. In step 220, light (preferably red or near-infrared) is emitted at the targeted region of the patient's brain. In step 230, the light therapeutically stimulates the targeted region of the patient's brain, preferably non-thermally and without damage to the region. The light can be applied in pulses or cycles to avoid damaging the targeted region of the brain, as well as avoid damage of the patient's skin or tissue between the light source and the targeted region.

It is contemplated that the light stimulation in the targeted region has a therapeutic effect. For example, in step 232, the light regulates neurobiological function at or related to the targeted region, for example provide neuroprotection against toxicity, improve frontal cortex (or other region's) oxygen consumption, and improve metabolic capacity in a region. In step 234, the light stimulation encourages neurotherapeutic effects at or around the targeted region, for example increased frontal cortex-based memory function. While in steps 236 and 238, the light forms temporary openings in the BBB or increases local perfusion in the targeted region, respectively. For example, when optional step 222 is applied to administer therapeutic exosomes to the patient in conjunction with light therapy, it is contemplated that either steps 236 or 238 occurs, or both, to help the therapeutic exosomes cross the BBB at the targeted region. In some embodiments, more than one light source is used to stimulate the targeted region or different regions of the patient's brain and perform steps 232, 234, 236, and 238.

### Example 1

Emerging data has demonstrated that depression involves inflammatory processes in the brain. Exosome treatments are thought to have potential anti-inflammatory benefits. This procedure intends to provide clinical relief for patients with depression by increasing localized perfusion to Brodmann area 25 (BA25) in combination with intravenous exosome delivery. The treatment uses neuro-navigated ultrasound to aid exosomal delivery to the subgenual cingulate (SGC, BA25). Patients undergoing treatment were both diagnosed with severe treatment-resistant Major Depressive Disorder (MDD) and had previously undergone transcranial magnetic stimulation (TMS) and intensive regimens of antidepressant medication with no relief of depressive symptoms. Functional and structural imaging was used to navigate ultrasound application targets for the SGC.

Two female patients (33, 51 years old) underwent a 30-minute targeted ultrasound session immediately prior to an intravenous exosome injection. The DWL Doppler Box Ultrasound was delivered using a 2 MHz probe at a power of 510 mW/cm². Using functional and structural neuroimaging, the DLPFC and SGC were navigated and targeted uniquely for each patient. Outcome measures, including the Global Rating Scale (GRC) and Beck's Depression Inventory (BDI-II), were administered before and after treatment.

Surprisingly, both patients were able to tolerate treatment without notable side effects. Both patients also surprisingly reported clinically meaningful improvement in their symptoms and had improved BDI scores after treatment (MΔ=10, SD=8.49). Advantageously, no adverse events were reported.

This treatment provides evidence supporting the safety and efficacy of combined exosome and focused ultrasound treatment for patients with MDD.

### Example 2

On the days of a near-infrared therapy session, patients will undergo 4-10 minutes of transcranial infrared laser stimulation (“TILS”). Laser dosage, duration of each session, and specific targets will depend on patient's condition. Specific targets will be determined for each patient through neuronavigation using MM scans done prior to these near-infrared therapy sessions. Both the patient and the near-infrared light administrators will wear protective eyewear; the administrators of the TILS will be careful not to shine the light in or near the eyes, and the patient's eyes will remain closed during the laser application.

The laser dose for the neurodegenerative dementia group will be a 3.4 W continuous laser wave, at a 1064 wavelength, with irradiance (power density) at 250 mW/cm². Laser stimulation will be alternated every minute between sites to prevent heating of the skin and to fractionate the dose for a total of 4 minutes per site (3.4 W×240 seconds=816 J/site). The total treatment duration will last 8 minutes per session, and will be repeated once a week for 5 weeks. For Alzheimer's, the site targeted will be the right prefrontal cortex, whereas Parkinson's patients will have laser delivered to the brain stem, bilateral occipital, parietal, temporal, or frontal lobes, or a combination thereof.

Patients with traumatic brain injury (TBI) will undergo a laser dose of 500 mW continuous wave LED source (mixture of 660 nm red and 830 nm NIR LEDs) with an irradiance of 22.2 mW/cm² (area of 22.48 cm²). Laser stimulation will be alternated every minute between sites to prevent heating of the skin and to fractionate the dose for a total of 5 minutes per site. The total treatment duration will last 10 minutes per session, and will be repeated once a week for 5 weeks.

The laser dose for patients with anxiety will be a 1 W 810 nm LED array applied to the forehead, whereas patients with depression will undergo a 810 nm laser (700 mW/cm2 and fluence of 84 J/cm²). The total treatment duration will last 5 minutes per session for both conditions, and will be repeated once a week for 6 weeks.

The device used for these near-infrared light therapy sessions is the Cytonsys CytonPro apparatus, though other appropriate devices are contemplated. CytonPro has pilot laser control, with a peak wavelength of 1064 nm, and a maximum optical (output) power of 10 W for the basic version, or 30 W for the ultra version. The maximum optical power density of CytonPro is 600 mW/cm², with an effective area of 4.5 cm in diameter. It also has flexible settings for power density, pulsing frequency, and treatment duration, allowing for real-time and accurate adjustments during the sessions.

Various objects, features, aspects, and advantages of the inventive subject matter will become more apparent from the following detailed description of preferred embodiments, along with the accompanying drawing figures in which like numerals represent like components.

The inventive subject matter provides apparatus, systems, and methods for comparative analysis of tissue and organ scans between patients or groups of patients without sensitivity to patient-specific or scanner specific characteristics, including prediction, diagnosis, prognosis, tracking, and treatment guidance.

The following description includes information that may be useful in understanding the present invention. It is not an admission that any of the information provided herein is prior art, necessary, or relevant to the presently claimed invention, or that any publication specifically or implicitly referenced is prior art.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of delivering an exosome into a patient's brain having a blood-brain barrier (BBB), comprising:
targeting a region of the patient's brain with a sonic transducer;
using the sonic transducer to sonicate the region of the patient's brain and facilitate selective delivery of the exosome to the region of the patient's brain; and
administering the exosome to the patient's blood stream, wherein the exosome traverses the BBB at the targeted region.

2. The method of claim 1, wherein the region of the patient's brain is associated with a disease condition.

3. The method of claim 2, wherein the disease condition is associated with a disease selected from the group consisting of dementia, a learning disorder, an anxiety disorder, a motor disorder, a consciousness disorder, a movement disorder, an attention disorder, a stroke, a vascular disease, Alzheimer's disease or other progressive potentially dementing conditions, Parkinson's disease, multiple sclerosis, cancer, schizophrenia, depression, anxiety disorder, developmental disorder, substance abuse, and traumatic brain injury.

4. The method of claim 1, wherein the region of the patient's brain is selected from the group consisting of frontal lobe, parietal lobe, occipital lobe, temporal lobe, hippocampus, hypothalamus, brain stem, cerebellum amygdala, corticospinal tract, thalamus, substantia nigra, basal ganglia, a tumor, a lesion, necrotic tissue, Heschl's gyms, Brodmann area 25, and a point of injury.

5. The method of claim 1, wherein the exosome is synthetic, derived from a full term placental tissue, or derived from an umbilical tissue.

6. The method of claim 1, further comprising delivering more than one exosome, of more than one type or derivation.

7. The method of claim 1, wherein the patient is human.

8. The method of claim 1, wherein the sonic transducer is an ultrasonic transducer.

9. The method of claim 1, wherein the region of the patient's brain is of therapeutic interest.

10. The method of claim 1, further comprising the step of administering a microbubble to the patient's blood stream.

11. The method of claim 10, further comprising the step of using the sonic transducer to sonicate the microbubble.

12. The method of claim 11, wherein the microbubble is at the targeted region of the patient's brain.

13. The method of claim 1, wherein the region of the patient's brain is sonicated for at least 10 minutes.

14. The method of claim 1, wherein selective delivery of the exosome to the region of the patient's brain is facilitated by (i) forming an opening in the BBB at the targeted region, or (ii) increasing local perfusion at the targeted region.

15. The method of claim 14, wherein the step of administering the exosome occurs after opening the BBB or increasing local perfusion at the targeted region.

16. The method of claim 1, wherein the sonic transducer is used at a power of at least 450 mW/cm².

17. The method of claim 1, wherein the opening in the BBB or increasing local perfusion is temporary.

18. The method of claim 1, further comprising administering a medication to the patient.

19. A method of treating a patient comprising delivering an exosome to a region of the patient's brain via at least one of (i) a temporary opening in the patient's BBB at the region or (ii) increasing local perfusion at the region.

20. The method of claim 19, further comprising the step of using a sonic transducer to (i) form the temporary opening or (ii) increase local perfusion at the region.

21. The method of claim 1, further comprising applying a light source to the region of the patient's brain.

22. The method of claim 21, wherein the light source is infrared or near-infrared.

23. The method of claim 21, wherein the light source is applied at least partially during sonication.

24. The method of claim 21, wherein the light source is applied before sonication.

25. A method of delivering an exosome into a patient's brain having a BBB, comprising:
targeting a region of the patient's brain with a light source;
emitting light from the light source at the targeted region of the patient's brain to facilitate selective delivery of the exosome to the region of the patient's brain; and
administering the exosome to the patient's blood stream, wherein the exosome traverses the BBB at the targeted region.

26. The method of claim 25, wherein the light source is infrared or near-infrared.

27. A method of therapeutically stimulating a region of a patient's brain, comprising:
targeting a region of the patient's brain with a light source; and
directing a light from the light source at the targeted region, wherein the light therapeutically stimulates the region of the patient's brain.

28. The method of claim 27, wherein the therapeutic stimulation is one of regulation of a neurobiological function in the region or a neurotherapeutic effect in the region.
